(19) United States
(12) Patent Application Publication
(10) Pub. No.: US 2009/0287056 A1
(43) Pub. Date: Nov. 19, 2009

(54) Device to facilitate suctioning of fluid during gastrointestinal endoscopy

(76) Inventor: Stanley F. Chang, Fresno, CA (US)

Correspondence Address: RICHARD A. RYAN;ATTORNEY AT LAW, 8497 N. MILLBROOK AVENUE, SUITE 101, FRESNO, CA, 93720, US

(21) Appl. No.: 12/152,167

(22) Filed: May 13, 2008

(51) Int. Cl. A61B 1/015

(52) U.S. Cl. 600/156

(57) ABSTRACT

A device and method to facilitate efficient suctioning of fluid from a gastrointestinal lumen, such as a colonic lumen, through a suction channel of an endoscope during gastrointestinal endoscopy, such as a colonoscopy. The device comprises an elongated body member having a bulbous member at its first end and a handle at its second end. The bulbous member is sized and configured to move through the suction channel and extend past the open distal end of the endoscope to engage mucosa and/or debris and prevent blockage of the orifice leading to the suction channel. The body member is stiff enough to push the bulbous member through the suction channel, yet sufficiently flexible to move inside the endoscope, and sized to interfere as little as possible with the suctioning of fluid. A plurality of projecting members can be used on the body member to substantially center it inside the suction channel.

### BACKGROUND OF THE INVENTION

1. Field of the Invention

The field of the present invention relates generally to devices and procedures for use in endoscopic examinations of the human gastrointestinal tract. More specifically, the device of the present invention relates to medical equipment and procedures used to overcome difficulties encountered while suctioning fluid during gastrointestinal endoscopic procedures, such as colonoscopy. Even more specifically, this invention relates to such devices that are configured to prevent blockage of the endoscope by mucosa and/or solid debris particles during endoscopic suctioning procedures.

2. Background

As used herein, the term “endoscope” or “scope” is used to refer to a endoscope, colonoscope, sigmoidoscope, proctoscope or other types of medical endoscopes. As known to those skilled in the art, a typical endoscope generally comprises a connecting tube, a handle and an insertion tube (the part inserted into the patient). In the text, the term “scope” generally refers to the insertion tube portion of the endoscope. In referring to the opposite ends of the scope or the guide of the present invention, the “proximal end” refers to that part of the scope or guide which is closest to the operator or physician endoscopist (hereinafter collectively referred to as “operator”) performing the procedure, and the “distal end” refers to that part of the scope or guide farthest from the operator. Although medically the rectum is generally not considered to be part of the colon, which includes the sigmoid colon, descending colon, transverse colon and ascending colon, for purposes of this disclosure general references herein to the term “colon” include the rectum. For purposes of simplifying this disclosure, the discussion and references herein are to use of the device of the present invention during a colonoscopy procedure to facilitate the operator with his or her examination of the colon. However, those skilled in the art of endoscopic procedures will readily understand that the device of the present invention is useful and readily adaptable for use with other endoscopy procedures, including but not limited to those in the gastrointestinal tract and peritoneal cavity.

In gastrointestinal endoscopy, particularly colonoscopy, there are a number of obstacles for the operator physician to overcome in order to achieve the objectives of the particular procedure. Colonoscopy is the most sensitive and specific means for examining the colon, particularly for the diagnosis of colon cancers and polyps. Because the cecum, the portion of the colon farthest from the anus, can be a common location for cancer, it is generally desirable that the entire colon be completely examined. During a typical colonoscopy procedure, the scope is inserted into the anus, through the rectum, and then advanced through the sigmoid colon, descending colon, transverse colon, ascending colon and into the cecum. In colonoscopy, as well as other gastrointestinal endoscopy, there is often fluid in the gastrointestinal lumen that needs to be suctioned out through the endoscope in order to obtain a clear view of the mucosal detail. Obtaining a clear view of the entire mucosa is paramount in colonoscopy so that the operator may detect small pre-malignant polyps, especially flat lesions, which may have an even higher malignant potential. As well known, the inability to completely clear the fluid from the gastrointestinal lumen compromises the quality of the examination.

Applying suction by pushing or otherwise engaging the suction button on the endoscope produces a vacuum in the scope's suction channel. This vacuum causes a negative pressure at the open distal end of the scope. The suction force is, of course, non-selective, meaning it cannot be made to selectively suck the fluid and not the colonic mucosa, or large solid debris in the lumen. Once a piece of colonic mucosa or large solid debris gets into and blocks the channel, the suctioning function immediately slows or ceases. As a result, clearing fluid residue from the interior surface of the colon is often quite difficult.

In general, the patient is not aware of any pain or other issues as a result of the mucosa being trapped by the suction through the endoscope. While trapping a piece of mucosa into the suction channel is innocuous to the patient, it is certainly a nuisance to the operator. The piece of entrapped mucosa has to be extricated from the open distal end of the scope by either temporarily removing the rubber valve on the handle of the scope to break the vacuum, or by injection of air or water through the suction channel. After clearing the piece of entrapped mucosa, suction can then resume, yet often only to have the same problem occur again and again. It is often, therefore, a frustrating and time consuming problem for the operator.

In general, suctioning of mucosa can be minimized by aligning the scope in a proper orientation in relation to the fluid, and simultaneous insufflation of air into the colon as suction is applied. However, no matter how great the skill of the colonoscopist, suctioning of mucosa is nevertheless a major challenge in the clearing of fluid from the gastrointestinal lumen due to the following: (1) fluid is always situated on the mucosal surface, never in the center of the colonic lumen; (2) the mucosal surface is not a hard and flat surface, instead it is soft, pliable and easily suctioned and usually located only millimeters away from the top of the fluid layer, and of course, immediately contiguous with the bottom of the fluid layer; (3) transverse or circumferential mucosal folds are almost always present in the gastrointestinal lumen and fluid usually collects in the trough between folds such that suctioning of the fluid also suctions the folds; and (4) peristalsis is a major source of difficulty in gastrointestinal endoscopy because a peristaltic wave obliterates the operator's view, requiring suctioning to stop immediately as the suctioning would suck in the mucosa.

The suctioning procedure is especially difficult when the fluid collection is located in an unfavorable position in relation to the position of the suction channel, such as when part of the mucosal surface is closer to the suction channel opening than the fluid. In this very common situation, when the pool of fluid and the position of the suction channel are not properly aligned, a piece of mucosa is often sucked into the endoscope in preference to the desired fluid. Typically, the operator must twist the scope in order to align the suction channel with the pool. Sometimes this is an arduous maneuver. After sucking out most of a pool of fluid, clearing the last thin layer of fluid can be quite difficult. Because this last bit of fluid is closest to the mucosa, it is also the hardest portion to clear.

Present preparation of the colon for colonoscopy has much improved by using a split-dose regimen to clean the colon of stool. For example, a dose of non-absorbable fluid such as polyethylene glycol is taken in the afternoon before the procedure, and the next dose is taken on the day of the colonoscopy, several hours before the procedure, along with a clear liquid diet. In general, this results in excellent clearing of stool from the colon. However, the trade-off for clearing the solid stool residue is that, very often, a large amount of fluid remains in the colon during the examination. This increased amount of fluid in the colon adds to the burden of having to suck fluid out of the gastrointestinal lumen. As a result, the entrapment of mucosa is usually more of a problem than solid debris.

Although generally less of a problem, blocking of the open distal end of the endoscope can also occur when a piece of solid or semi-solid stool debris is suctioned into the channel and blocks passage of fluid through the channel. The debris must be expelled by injecting air or water through the channel to clear the channel for further suctioning. If water needs to be injected into the channel, then this additional water will need to be sucked out, which adds to the problem by increasing the likelihood of the mucosa being entrapped.

Various types of catheter-like instruments, which are passed through the suction channel of the endoscope, have been utilized in gastrointestinal endoscopy. The most common of these devices are biopsy forceps and snares. These and other such devices are configured to pass through the suction channel and exit the tip of the scope to perform their function(s) in the colon lumen. Such devices necessarily have to have a shaft that is large enough to house the functioning components of the snare, forceps or other instrument. The resulting diameter or width of the shaft of such instruments leaves little room between the inside wall of the suction channel and the outside wall of the shaft for suctioning fluid from inside the gastrointestinal lumen. In fact, with one of these instruments in place, moving the fluid through the long channel of the insertion tube of the scope, which is typically 160 cm long, is extremely inefficient, if not impossible. In addition, the tip of some devices, such as the polypectomy snare utilized in colonoscopy, is typically pointed and, as such, would pose a hazard for the mucosal surface of the colon if the device were utilized to push against the colon to prevent the mucosa from being sucked into the colonoscope. The existence of a pointed tip is also a problem with attempting to use a balloon catheter, in addition to the instrument's cost, as a device to push against the mucosa.

A transparent cap has also been designed to fit onto the tip of the scope. This cap can push the mucosa away from the tip of the scope. However, it is inconvenient to use because it has to be preloaded onto the tip of the scope before the scope is inserted into the patient and it cannot be taken off the scope without removing the entire scope from the patient. In addition, it cannot prevent large solid particles from being sucked into and blocking the suction channel. Furthermore, because the cap is located at the distal end of the scope, it can compromise the operator's view of the colon.

Suctioning fluid to clear the view of the colon at the present state of the art remains a time-consuming and arduous part of colonoscopy. In fact, conscientious colonoscopists commonly spend 20% to 30% of the colonoscopy time suctioning residual fluid from the colon in order to gain a complete and generally unobscured view thereof. What is needed, therefore, is a device which helps the operator suction fluid from inside the gastrointestinal lumen through the channel of an endoscope without the open distal end of the scope being plugged by mucosa and/or debris. Such a device does not exist at this time. In fact, to the inventor's knowledge, there has never been an instrument configured to specifically solve the problem of preventing mucosa and debris from blocking the suction channel of the scope while allowing efficient suction removal of fluid from the gastrointestinal lumen through the scope. Such a device should be configured to be effective at preventing blockage of the suction channel of an endoscope by mucosa and/or debris at the open distal end of the scope while allowing efficient removal of fluid through the suction channel. Use of the device should simplify gastrointestinal endoscopy procedures, particularly colonoscopy, and reduce the operator labor and time presently associated with colonoscopies and other endoscopic procedures. Preferably, the device should be relatively inexpensive to manufacture and easy to use.

### SUMMARY OF THE INVENTION

The device for facilitating suctioning of fluid during gastrointestinal endoscopy of the present invention provides the benefits and solves the problems identified above. That is to say, the present invention discloses a device which facilitates suctioning of fluid from inside an gastrointestinal lumen through the suction channel of an endoscope by preventing blockage of the open distal end of the scope. The device of the present invention is sized and configured to be inserted through the suction channel of the scope to prevent mucosa and/or debris from blocking the channel so as to facilitate suctioning of fluid from the gastrointestinal lumen. The device is configured to effectively prevent the blockage of the open distal end of the scope while allowing fluid to be efficiently suctioned through the suction channel of the scope. The device of the present invention is easy to use and reduces the time associated with a colonoscopy or other gastrointestinal endoscopy by substantially eliminating the likelihood that mucosa and/or debris will block the open end of the suction channel of the scope utilized to remove fluid from the gastrointestinal lumen. As such, the device is likely to improve the examination of the gastrointestinal tract by providing more effective and easier removal of fluid therefrom. In its preferred embodiment, the device of the present invention is relatively inexpensive to manufacture.

In one embodiment of the present invention, the device comprises an elongated body member having a distally disposed first end and a proximally disposed second end, a bulbous member at the first end of the body member and a handle at the second end of the body member. The bulbous member is sized and configured to be pushed into and through the suction channel of an endoscope. Preferably, the bulbous member has a smooth, curved surface so that it will not tear or otherwise damage the mucosa of the gastrointestinal lumen from which fluid is being suctioned. The bulbous member can be configured in a round, teardrop, capsule or other shape. The body member is made out of a material and sized such that is sufficiently stiff to allow the operator to push the bulbous member through the suction channel, sufficiently flexible to follow the sometimes tortuous path of the endoscope inside the gastrointestinal tract and of sufficient length to extend the bulbous member past the open distal end of the endoscope. The diameter or width of the body member is selected to provide as much space as possible between the outer surface of the body member and the interior surface of the endoscope to allow fluid to pass in a manner that facilitates efficient suctioning of the fluid from the gastrointestinal lumen. In the preferred embodiment, the handle is sized and configured to prevent full entry of the device into the suction channel. Preferably, the handle is sized and configured to prevent entry of the handle into the suction channel. In an alternative embodiment, the handle is a continuation of the body member that is made long enough to allow the user to grab onto the proximal aspect of thereof and direct and manipulate the device without risk of the body member completely entering the suction channel. Alternatively, the device can have an enlarged member, such as a plate, knot, clip-on member or other components, at the proximal end of the body member that prevents complete entry into the suction channel. In the preferred embodiment, the device is integrally formed and made out of a human-use suitable plastic, metal or composite material. In one alternative embodiment, the handle is a separate component that is attached to the second end of the body member.

During use, the bulbous member is inserted into the suction channel and then pushed along the suction channel, either by pushing on the handle and/or the body member, until it extends beyond the open distal end of the endoscope. Once in position, the operator initiates suctioning of the fluid from the gastrointestinal lumen. Any mucosa or larger size debris that would otherwise be likely to block the orifice leading into the suction channel at the distal end of the endoscope will be prevented from blocking the suction channel by being engaged against the bulbous member. Because of the small diameter of the body member, fluid will still be able to efficiently suctioned out of the gastrointestinal lumen through the suction channel. By preventing mucosa and/or debris from blocking the suction channel, the device of the present invention reduces the time and effort required to suction fluid from the gastrointestinal lumen so that the operator may more effectively conduct his or her examination.

Accordingly, the primary objective of the present invention is to provide a device to facilitate suctioning fluid during gastrointestinal endoscopy that provides the advantages discussed above and which overcomes the disadvantages and limitations associated with presently available devices and methods for suctioning fluid during gastrointestinal endoscopy procedures.

It is also an important objective of the present invention to provide a device that facilitates suctioning of fluid from a gastrointestinal lumen through the suction channel of an endoscope by preventing blockage of the channel by mucosa and/or debris during a gastrointestinal endoscopy procedure, such as colonoscopy.

It is also an important objective of the present invention to provide a device that is sized and configured to be inserted through the suction channel of an endoscope to prevent mucosa and/or debris from blocking a suction channel in the scope so as to facilitate suctioning of fluid from a gastrointestinal lumen.

It is also an important objective of the present invention to provide a device which is configured to effectively prevent the blockage of the open distal end of an endoscope while allowing fluid to be efficiently suctioned through the suction channel of the scope.

It is also an important objective of the present invention to provide a device that is easy to use and reduces the time associated with a colonoscopy or other gastrointestinal endoscopy by preventing mucosa and/or debris from blocking the open distal end of the endoscope utilized to remove fluid from the gastrointestinal lumen.

It is also an important objective of the present invention to provide a device that facilitates suctioning of fluid from a gastrointestinal lumen through a channel in an endoscope that comprises an elongated body member having a bulbous configuration at the first or distal end that is sized to fit through the channel and prevent mucosa and/or debris from blocking the channel.

The above and other objectives of the present invention are explained in greater detail by reference to the attached figures and description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a prior art illustration of a colonoscope advancing through the colon to perform a colonoscopy procedure;

FIG. 2 is a prior art illustration of a portion of the mucosa sucked into the open distal end of a colonoscope, thereby blocking the suction channel and preventing fluid from being suctioned through the channel;

FIG. 3 is a prior art illustration of piece of debris sucked into the open distal end of a colonoscope, thereby blocking the suction channel and preventing fluid from being suctioned through the channel;

FIG. 4 is a side view of a device to facilitate suctioning of fluid during gastrointestinal endoscopy configured according to a preferred embodiment of the present invention;

FIG. 5 is a cross-sectional view illustrating use of the device of FIG. 4 inside an endoscope to prevent blockage of the suction channel by mucosa and debris so as to facilitate suctioning of fluid from inside the gastrointestinal lumen;

FIG. 6 is a side view of an alternative embodiment of the device of the present invention shown with a lengthened body member as the handle for manipulation of the device and to prevent full entry of the device into the suction channel of the endoscope; and

FIG. 7 is a side view of another alternative embodiment of the device of the present invention shown with an enlarged member at the distal end of the body member to prevent full entry of the device into the suction channel of the endoscope.

### DETAILED DESCRIPTION OF THE PREFERRED EMBODIMENTS

With reference to the figures where like elements have been given like numerical designations to facilitate the reader's understanding of the present invention, the preferred embodiments of the present invention are set forth below. The enclosed text and drawings are merely illustrative of a preferred embodiment and represent one of several different ways of configuring the present invention. Although specific components, materials, configurations and uses are illustrated, it should be understood that a number of variations to the components and to the configuration of those components described herein and in the accompanying figures can be made without changing the scope and function of the invention set forth herein. For instance, although the figures and description provided herein are directed to use of the device of the present invention during a colonoscopy to suction fluid from the colon, those skilled in the art will readily understand that this is set forth merely for purposes of simplifying the present disclosure and that the present invention is not so limited.

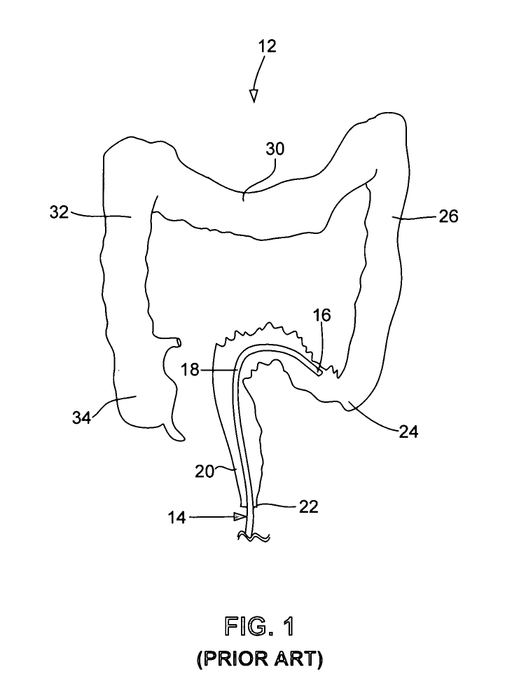

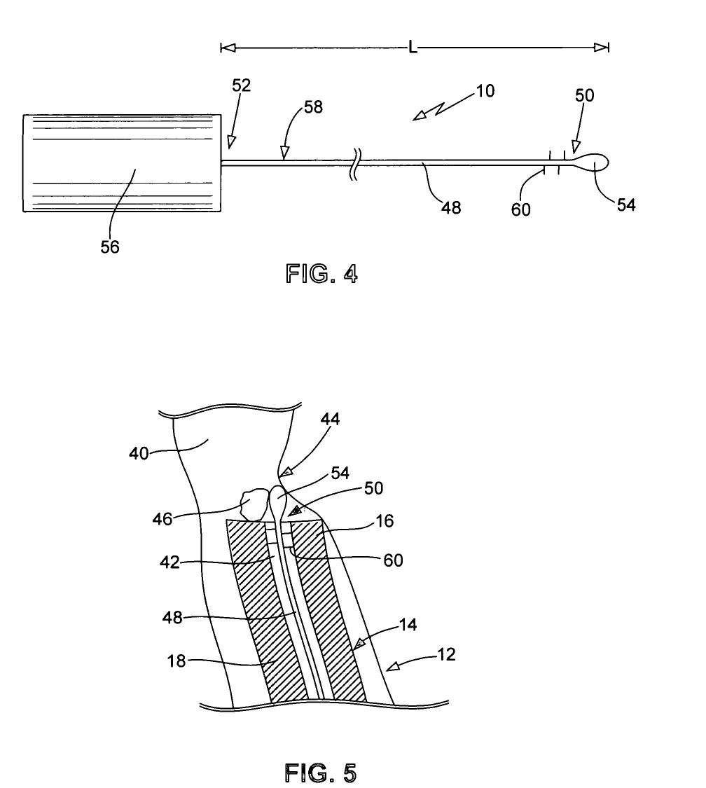

A device to facilitate the suctioning of fluid during a gastrointestinal endoscopy that is manufactured out of the components and configured pursuant to a preferred embodiment of the present invention is shown generally as 10 in FIGS. 4 through 7. As set forth in more detail below, device 10 is utilized to assist in the removal of fluid during a colonoscopy, as an example of a gastrointestinal endoscopy procedure, to benefit the investigation of the colon 12, shown in its entirety in FIG. 1, with a colonoscope 14 or equivalent medical device shown therein. The distal end or tip 16 and insertion tube or body 18 of colonoscope 14 are introduced into the rectum 20 through the anus 22 and then into the sigmoid colon 24. After negotiating the loop in the sigmoid colon 24, the colonoscope 14 enters the descending colon 26, advances through the transverse colon 30 and the ascending colon 32 and into the cecum 34. After completing the colonoscopy procedure, the colonoscope 14 is withdrawn and then removed from the patient.

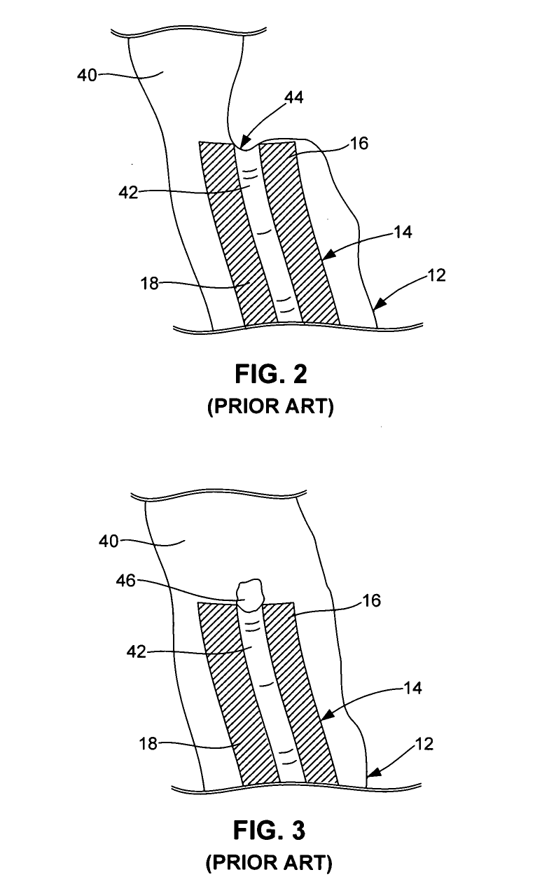

As well known to those skilled in the art, the typical colonoscope 14 has three or more channels disposed inside insertion tube 18 that are each utilized for one or more different functions. For purposes of simplifying the present drawings, however, the colonoscope 14 shown in use inside colonic lumen 40 in FIGS. 2, 3 and 5 illustrates only the suction channel 42 through which fluid is suctioned from colonic lumen 40. In addition to suction channel 42, colonoscope 14 typically includes a light channel for carrying light to illuminate the area ahead of distal end 16 inside colonic lumen 40 and an air/water channel to allow the operator to insufflate air and to clean the lens with water inside colonic lumen 40. Suction channel 42 is also utilized as the scope's instrument channel, through which various instruments, including but not limited to forceps and snares, are inserted for use inside colonic lumen 40, for colonoscope 14.

During a colonoscopy, it is not unusual for the suction channel 42 to become blocked at or near the open distal end 16 of endoscope 14 while the operator is trying to suction fluid out of the colonic lumen 40 to obtain a better view of the colonic lumen's interior wall. Examples of two problems that are likely to occur are illustrated in FIGS. 2 and 3, both of which are known to block the suction channel 42 and prevent the suctioning of fluid from the colonic lumen 40. FIG. 2 illustrates a portion of the colonic wall or mucosa, shown as 44, having been sucked against and partially into suction channel 42 at the distal end 16 of colonoscope 14. FIG. 3 illustrates a piece of debris 46 having been sucked against and partially into suction channel 42 at the distal end 16 of colonoscope 14. In both circumstances, the suction channel 42 at open distal end 16 is either completely or substantially blocked, thereby preventing any further suctioning of fluid from inside colonic lumen 40 or making suction difficult. As set forth in the background, the operator can attempt to dislodge the stuck mucosa 44 or the debris 46 from the suction channel 42 by shutting off the suction action in an attempt to release the mucosa 44 or debris 46 and, if that does not work, injecting air or water into the suction channel 42 to force the mucosa 44 or debris 46 away from the open distal end 16 of colonoscope 14. After releasing the mucosa 44 or debris 46, the mucosa 44 or debris 46 may become re-engaged with suction channel 42 at the distal end 16 upon re-initiation of the suctioning action. In addition, if water or other liquid is utilized to force the mucosa 44 or debris 46 away from the suction channel 42, this results in more liquid for the operator to remove in order to obtain the desired view of the interior surface of the colon 12.

As shown in FIG. 5, device 10 of the present invention is configured to prevent the mucosa 44 and/or debris 46 from blocking the suction channel 42 at the open distal end 16 of the colonoscope 14 while suctioning fluid from inside the colonic lumen 40. The device 10 comprises an elongated body member 48 having a first or distal end 50 and second or proximal end 52, as best shown in FIG. 4. The body member 48 is configured as a long, thin shaft. A generally bulbous member 54 is located at the first end 50 and a handle 56 is located at the second end 52. These components work together to prevent the mucosa 44 and debris 46 from blocking the suction channel 42 at or near the distal end 16 of colonoscope 14, as shown in FIG. 5, while allowing efficient suctioning of fluid from inside the colonic lumen 40.

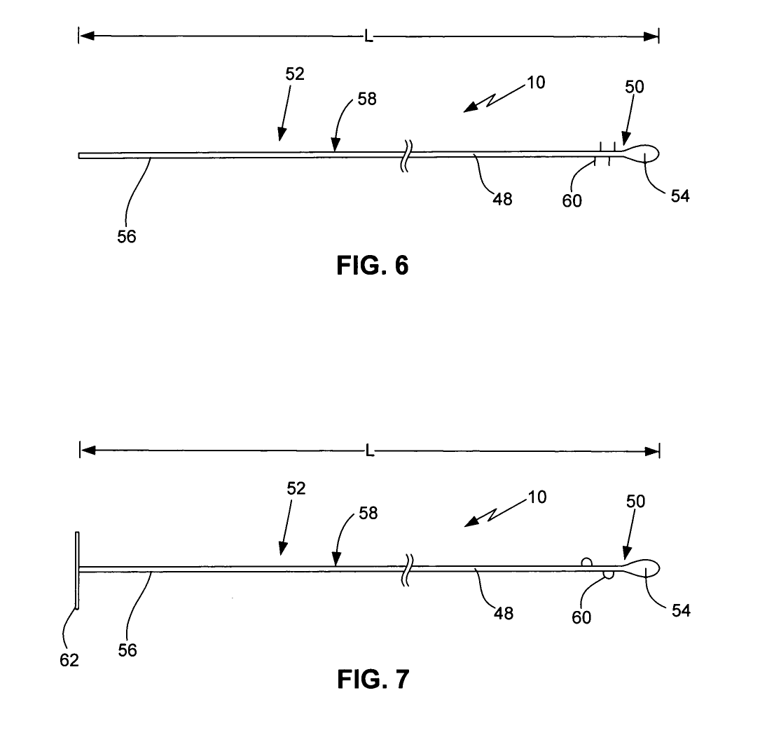

In the preferred embodiment, the elongated body member 48 is configured to be as thin as possible to provide room inside suction channel 42 for suctioning fluid therethrough, flexible enough to follow the curved path of the colonoscope 14 inside the colon 12 and stiff enough to push through the long suction channel 42 of colonoscope 14. In the preferred embodiment, the body member 48 is made from a solid material, such that it does not have a lumen. As known in the art, existing cannulas require a lumen to contain the operating components of the instrument. Because of the lumen size, these instruments occupy much of the suction channel 42, resulting in inefficient (if even possible) suctioning of fluid from colonic lumen 40 through suction channel 42. Eliminating the lumen generally allows a much smaller diameter body member 48 than would otherwise be possible, thereby providing as much room as possible between the interior wall of suction channel 42 and the outer surface 58 of body member 48 to facilitate effective and efficient suctioning of fluid from inside colonic lumen 40 through suction channel 42. The typical length of colonoscope 14 is 160 cm to 165 cm, with an additional 15 cm to 20 cm between the port of entry at the handle and the beginning of the scope 14, for a total length of approximately 175 cm. It is preferred that the length of body member 48 of device 10 be selected such that the length L, shown in FIGS. 4 through 7, from the proximal end 52 of body member 48 to the distal tip of bulbous member 54 be longer than the length of the colonoscope 14 so bulbous member 54 can extend past the distal end 16 of colonoscope 14. In the embodiment that includes a separate handle 56, shown in FIG. 4, typically a length L of approximately 190 cm will be sufficient. In an embodiment without a separate handle 56, such as shown in FIGS. 6 and 7, the length L of body member 48 may have to be longer to prevent its full entry into the suction channel 42. The body member 48 can be manufactured out of a wide variety of materials, such as plastic, metal and composites, that are selected for their maneuverability, stiffness, strength and suitability for use in the human body.

In the preferred embodiment, the bulbous member 54 is configured as a smooth, curved tip on device 10. The bulbous member 54 can have a shape that is generally round, teardrop, capsule or conical, as well as a variety of other like configurations. Generally, bulbous member 54 is relatively short. To fit through suction channel 42, bulbous member 54 must have a width or diameter that is slightly smaller than the diameter of the suction channel 42, which is typically approximately 3 mm. To best achieve the prevention of blockage objectives of the present invention, preferably the width/diameter of bulbous member 54 is only slightly less than the diameter of suction channel 42. To avoid any damage to the mucosa 44, it is preferred that the distal end of bulbous member 54 not have any projections that could possibly pierce or otherwise damage mucosa 44 and that bulbous member 54 not have any sharp edges.

In the embodiment with a separate handle 56, the handle 56 at the second end 52 of body member 48 is sized and configured to prevent full entry of the device 10 into the suction channel 42. In a preferred configuration, handle 56 has a circular cross-section with a diameter large enough to prevent passage into suction channel 42. Preferably, handle 56 is also sized and configured to be easy for the operator to grasp and to allow him or her to beneficially manipulate device 10 so that the bulbous member 54 may be pushed along suction channel 42 to and through the open distal end 16 of the colonoscope 14. If desired, the exterior of handle 56 may be coated, covered or integrally formed with a gripping surface to improve the operator's grasp of handle 56 and manipulation of device 10. In alternative embodiments of the present invention, examples of which are shown in FIGS. 6 and 7, handle 56 is continuation of body member 48. In these embodiments, the proximal aspect of body member 48 functions as the handle 56 that the operator grasps to direct and manipulate the body member 48 to place bulbous member 54 into position so that it will prevent mucosa 44 and debris 46 from blocking the orifice into the suction channel 42 at the distal end 16 of the colonoscope 14. FIG. 7 shows an embodiment that utilizes an enlarged member 62, such as the plate shown, that is sized and configured to prevent full entry of device 10 into suction channel 42. In other embodiments, enlarged member 62 can be a knot tied in body member 48, a clip-on object removably attached to the proximal aspect of the body member 48 and/or other components that prevent full entry of device 10 into body member 48. The use of a longer length body member 48 as handle 56 of FIGS. 6 and 7 is believed to be less expensive to manufacture than the handle 56 of FIG. 4. The use of a knot, clip-on object or like component is a relatively inexpensive way to prevent full entry of device 10 into suction channel 42.

In one of the preferred embodiments, body member 48, bulbous member 54 and handle 56 are integrally formed. In another embodiment, only body member 48 and bulbous member 54 are integrally formed and handle 56 is attached to the second end 52 of body member 48. In yet another embodiment, each of the body member 48, bulbous member 54 and handle 56 are separately formed and then attached or otherwise joined together to form a single unit. In yet another embodiment, separate handle 56 does not exist. Instead, the body member 48 is long enough to be prevented from completely entering the suction channel 42 (as set forth above).

The preferred embodiment also comprises a plurality of small, outwardly extending projecting members 60 at or near the first end 50 of body member 48. Preferably, the projecting members 60 are integrally formed with body member 48 or are very securely attached to the outer surface 58 thereof and are configured to be relatively short and generally perpendicular to body member 48. The projecting members 60 can be hair-like, bead-like (as shown in FIG. 7) or a combination of both and/or other components. The purpose of the projecting members 60 is to substantially dispose and maintain the body member 48 at or near the center of suction channel 42 to provide as much room as possible on both sides thereof for fluid and smaller debris to be sucked through the suction channel 42 without interference from device 10 so as to allow efficient suctioning of fluid from the colonic lumen 40. Projecting members 60 prevent body member 48 from moving to one side and, therefore, leaving a wide space at the opposite side of the suction channel 42. In one embodiment, shown in FIGS. 4 to 6, the projecting members 60 are substantially hair-like and are positioned at or near the junction of the first end 50 of body member 48 and bulbous' member 54. In FIG. 7, projecting members 60 are bead-like shaped projections. In the preferred embodiments, projecting members 60 are positioned near the orifice at the distal end 16 of colonoscope 14. At that location, the projecting members 60 can serve as a filter to block the larger pieces of solid debris 46, while still allowing the fine residual solid particles to pass through. If desired, the projecting members 60 can extend around the outer surface 58 of body member 48 in a spiral or like pattern (i.e., with a 120 degree spacing) and be located elsewhere along the length of body member 48.

In use, device 10 is inserted into suction channel 42 at the valve opening of the suction channel 42, typically located on the handle of colonoscope 14. While grasping body member 48 (at first) of device 10, the operator pushes to move the bulbous member 54 and body member 48 through the suction channel 42 to the open distal end 16 of colonoscope 14. The bulbous member 54 is then pushed, by pushing on the body member 48 and/or handle 56, such that the bulbous member 54 protrudes slightly beyond the open distal end 16 of the scope 14. As suction is applied, fluid flows from the colonic lumen 40, through the orifice at the distal end 16 and into suction channel 42, while the mucosa 44 is held back and prevented from blocking the open distal end 16 of colonoscope 14, as shown in FIG. 5. The smooth, rounded surface of bulbous member 54 allows atraumatic pushing of the mucosa 44 away from the open distal end 16 of the scope 14. As also shown in FIG. 5, debris 46 is also prevented from entering the suction channel 42 when suctioning is applied to remove fluid from the colonic lumen as body member 48 is kept at the center of the suction channel 42 at or near the distal end 16 by projecting members 60. In summary, fluid will be suctioned into the suction channel 42 and out of the colonic lumen 40 while mucosa 44 and any potentially obstructing debris 46 will be prevented from entering suction channel 42. The amount that bulbous member 54 extends beyond the distal end 16 of the scope 14 is adjustable by pushing or pulling the body member 48 or the handle 56 of device 10. For example, when suction is not being applied, the distal aspect of device 10 can be withdrawn into the suction channel 42 of the scope 14, until needed for suctioning fluid from the colonic lumen 40 so that it does not obstruct the view of the mucosa 44. The projecting members 60 will generally center the body member 48 inside the suction channel 42 at or near distal end 16 to provide as much space as possible inside suction channel 42 so that fluid from the colonic lumen 40 can be efficiently suctioned therefrom. The device 10 of the present invention can be used during suctioning procedures, removed from the suction channel 42 when necessary to use the suction channel 42 for other endoscopic instruments, such as forceps and snares, and then reinserted into the suction channel 42 after the other instruments are removed for further suctioning, as desired by the operator.

While there are shown and described herein certain specific alternative forms of the invention, it will be readily apparent to those skilled in the art that the invention is not so limited, but is susceptible to various modifications and rearrangements in design and materials without departing from the spirit and scope of the invention. In particular, while the above description contains many specifics, these should not be construed as limitations on the scope of the invention, but rather as an exemplification of one or more preferred embodiments thereof. Further, it should be noted that the present invention is subject to modification with regard to assembly, materials, size, shape and use. Some of the components described herein can be made integral with each other to reduce the number of separate components.

What is claimed is:

1. A device to facilitate suctioning of fluid from a gastrointestinal lumen through a suction channel disposed in an endoscope having an open distal end, said device comprising:
an elongated body member having a distally disposed first end and a proximally disposed second end; and
a bulbous member at said first end of said body member, said bulbous member sized and configured to be pushed into and through said suction channel, said body member having a length configured to extend said bulbous member beyond said open distal end of said endoscope to engage mucosa and/or debris in said gastrointestinal lumen and prevent blockage of said suction channel.

2. The device according to claim 1 further comprising a handle at said second end of said body member.

3. The device according to claim 2, wherein said handle is sized and configured to prevent full entry of said device into said suction channel.

4. The device according to claim 2, wherein said handle is defined by the distal aspect of said body member.

5. The device according to claim 4 further comprising an enlarged member at or near the second end of said body member.

6. The device according to claim 1, wherein said bulbous member is configured to safely engage said mucosa.

7. The device according to claim 1 further comprising a plurality of projecting members on said body member, said projecting members configured to generally dispose said body member at or near the center of said suction channel.

8. The device according to claim 7, wherein said projecting members are disposed at or near the first end of said body member.

9. The device according to claim 1, wherein said body member is both sufficiently stiff to push said bulbous member through said suction channel and sufficiently flexible to move inside said endoscope.

10. A device to facilitate suctioning of fluid from a gastrointestinal lumen through a suction channel disposed in an endoscope having an open distal end, said device comprising:
an elongated body member having a distally disposed first end and a proximally disposed second end;
a bulbous member at said first end of said body member, said bulbous member sized and configured to be pushed through said suction channel; and
a handle at said second end of said body member, said handle sized and configured so as to prevent entry of said handle into said suction channel, said body member having a length selected to extend said bulbous member beyond said open distal end of said endoscope when said handle is prevented from entry into said suction channel.

11. The device according to claim 10, wherein said bulbous member is configured to safely engage said mucosa.

12. The device according to claim 10 further comprising a plurality of projecting members on said body member, said projecting members configured to generally dispose said body member at or near the center of said suction channel.

13. The device according to claim 12, wherein said projecting members are disposed at or near the first end of said body member.

14. A method of suctioning fluid from a gastrointestinal lumen through a suction channel disposed in an endoscope having an open distal end, the method comprising the steps of:
a) inserting a bulbous member into said suction channel, said bulbous member at a first end of an elongated body member;
b) advancing said bulbous member through said suction channel to said distal end of said endoscope;
c) extending said bulbous member beyond said distal end of said endoscope;
d) initiating suctioning of fluid from said gastrointestinal lumen through said suction channel and along said body member; and
e) engaging said bulbous member against mucosa and/or debris in said gastrointestinal lumen as necessary to prevent blockage of said suction channel.

15. The method according to claim 14 further comprising a handle at a second end of said body member.

16. The method according to claim 15, wherein said handle is sized and configured to prevent full entry of said device into said suction channel.

17. The method according to claim 14, wherein said bulbous member is configured to safely engage said mucosa.

18. The method according to claim 14, wherein said body member has a plurality of projecting members disposed thereon, said projecting members configured to generally dispose said body member at or near the center of said suction channel.

19. The method according to claim 18, wherein said projecting members are disposed at or near the first end of said body member.

20. The device according to claim 14, wherein said body member is both sufficiently stiff to push said bulbous member through said suction channel and sufficiently flexible to move inside said endoscope.
